(19) United States
(12) Patent Application Publication
(10) Pub. No.: US 2023/0248810 A1
(43) Pub. Date: Aug. 10, 2023

(54) METHOD FOR TREATING ALZHEIMER'S DISEASE BY TARGETING MAPT GENE

(71) Applicant: Modalis Therapeutics Corporation, Tokyo (JP)

(72) Inventors: Iain Robert THOMPSON, Waltham, MA (US); Tetsuya YAMAGATA, Waltham, MA (US); Talha AKBULUT, Waltham, MA (US)

(73) Assignee: Modalis Therapeutics Corporation, Tokyo (JP)

(21) Appl. No.: 18/004,626

(22) PCT Filed: Jul. 9, 2021
(86) PCT No.: PCT/JP2021/025974

§ 371 Date: Jan. 6, 2023

Related U.S. Application Data
(60) Provisional application No. 63/049,736, filed on Jul. 9, 2020.
(60) Provisional application No. 63/212,429, filed on Jun. 18, 2021.

(51) Int. Cl. A61K 38/46; A61K 31/7088; C07K 14/47; C12N 15/86; C12N 9/22; C12N 15/11

(52) U.S. Cl. CPC A61K 38/465; A61K 31/7088; C07K 14/4703; C12N 15/86; C12N 9/22; C12N 15/11; C12N 2750/14143; C12N 2750/14171; C12N 2310/20; C12N 2800/80

(57) ABSTRACT

A polynucleotide, comprising the following base sequences: (a) a base sequence encoding a fusion protein of a nuclease-deficient CRISPR effector protein and a transcription repressor, and (b) a base sequence encoding a guide RNA targeting a continuous region of 18 to 24 nucleotides in length in a region set forth in SEQ ID NO: 54, 55, 56, 57, 68, 153 or 97 in the expression regulatory region of human MAPT gene. are expected to be useful for treating or preventing tauopathy including Alzheimer's disease.

### TECHNICAL FIELD

The present invention relates to methods for treating Alzheimer's disease by targeting the human microtuble-associated protein tau (MAPT) gene, and the like. More particularly, the present invention relates to methods and agents for treating or preventing Alzheimer's disease by suppressing expression of human MAPT gene by using a guide RNA targeting a particular sequence of human MAPT gene and a fusion protein of a transcription inhibitor and a CRISPR effector protein, and the like.

### BACKGROUND ART

Tau protein is a microtubule-binding protein that is mainly expressed in the nervous system. It promotes polymerization of tubulin, stabilizes microtubules, and contributes to the construction and maintenance of nerve axons. Tau protein is a product of a single gene named MAPT (microtubule-associated protein tau) located on chromosome 17 in humans, and six kinds of isoforms are expressed in the human brain by alternative splicing. All of these isoforms are known to lose binding ability to microtubule and self-aggregate when excessively phosphorylated. Self-assembly of Tau protein is involved in pathologies such as Alzheimer's disease (hereinafter to be referred to as AD) and frontotemporal dementia with Parkinsonism linked to chromosome (hereinafter to be referred to as FTDP-17). Aggregates of phosphorylated tau are generated in nerve cells in the brain and contribute to many neurodegenerative diseases.

Thus, a neurodegenerative disease accompanied by aggregation and intracellular accumulation of tau and considered to involve tau aggregation process in the onset of the disease is called Tauopathy.

A plurality of therapeutic strategies has been proposed to treat AD (non-patent document 1), and the gene therapy approach has been attracting attention as one of the strategies.

As a gene therapy targeting MAPT, for example, WO2018/102665 A1 discloses an invention directed to a genetic modulator of a MAPT gene, comprising a DNA-binding domain that binds to a target site of at least 12 nucleotides in the MAPT gene; and a transcriptional regulatory domain or nuclease domain.

On the other hand, a system using a combination of Cas9 with deactivated nuclease activity (dCas9) and a transcription activation domain or transcription repression domain has been developed in recent years, in which expression of a target gene is controlled through targeting of the protein to the gene by using guide RNA and without cleaving DNA sequence of the gene (patent document 1, which is incorporated herein by reference in its entirety). Its clinical application is expected (non-patent document 2, which is incorporated herein by reference in its entirety). However, a problem exists in that a sequence encoding a complex of dCas9, guide RNA and a co-transcription repressor exceeds the capacity of the most common viral vectors (e.g., AAV), which represent the most promising method for gene delivery in vivo (non-patent document 3, which is incorporated herein by reference in its entirety).

### CITATION LIST

### Patent Literature

[PTL 1] WO2013/176772

### Non Patent Literature

[NPL 1] Ballard C. et al., Lancet 2011; 377:1019-31
[NPL 2] Dominguez A. et al., Nat Rev Mol Cell Biol. 2016 January; 17(1): 5-15
[NPL 3] Liao H. et al., Cell. 2017 Dec. 14; 171(7): 1495-507

### SUMMARY OF INVENTION

### Technical Problem

Accordingly, it is one object of the present invention to provide novel therapeutic approaches to tauopathy (particularly, AD).

This and other objects, which will become apparent during the following detailed description, have been achieved by the inventors' discovery that the expression of human MAPT gene (Gene ID: 4137) can be strongly suppressed by using a guide RNA targeting a particular sequence of human MAPT gene and a fusion protein of a transcription repressor and a nuclease-deficient CRISPR effector protein.

### Solution to Problem

The present inventors have found that the expression of human MAPT gene can be strongly suppressed by a single AAV vector carrying a base sequence encoding the fusion protein and a base sequence encoding the guide RNA, using a compact nuclease-deficient CRISPR effector protein and a compact transcription repressor.

Thus, the present invention provides:

[1] A polynucleotide, comprising the following base sequences:

(a) a base sequence encoding a fusion protein of a nuclease-deficient CRISPR effector protein and a transcription repressor, and

(b) a base sequence encoding a guide RNA targeting a continuous region of 18 to 24 nucleotides in length in a region set forth in SEQ ID NO: 54, 55, 56, 57, 68, 153 or 97 in the expression regulatory region of human MAPT gene.

[2] The polynucleotide of [1], wherein the base sequence encoding the guide RNA comprises the base sequence set forth in SEQ ID NO: 54, 55, 56, 57, 68, 153 or 97, or the base sequence set forth in SEQ ID NO: 54, 55, 56, 57, 68, 153 or 97 in which 1 to 3 bases are deleted, substituted, inserted, and/or added.

[3] The polynucleotide of [1] or [2], comprising at least two different base sequences encoding the guide RNA.

[4] The polynucleotide of any of [1] to [3], wherein the transcriptional repressor is selected from the group KRAB, MeCP2, SIN3A, HDT1, MBD2B, NIPP1, and HP1A.

[5] The polynucleotide of [4], wherein the transcriptional repressor is KRAB.

[6] The polynucleotide of any of [1] to [5], wherein the nuclease-deficient CRISPR effector protein is dCas9.

[7] The polynucleotide of [6], wherein the dCas9 is derived from Staphylococcus aureus.

[8] The polynucleotide of any of [1] to [7], further comprising a promoter sequence for the base sequence encoding the guide RNA and/or a promoter sequence for the base sequence encoding the fusion protein of the nuclease-deficient CRISPR effector protein and the transcriptional repressor.

[9] The polynucleotide of [8], wherein the promoter sequence for the base sequence encoding the guide RNA is selected from the group U6 promoter, SNR6 promoter, SNR52 promoter, SCR1 promoter, RPR1 promoter, U3 promoter, and H1 promoter.

[10] The polynucleotide of [9], wherein the promoter sequence for the base sequence encoding the guide RNA is U6 promoter.

[11] The polynucleotide of any of [8] to [10], wherein the promoter sequence for the base sequence encoding the fusion protein of the nuclease-deficient CRISPR effector protein and the transcriptional repressor is a ubiquitous promoter or a neuron specific promoter.

[12] The polynucleotide of [II], wherein the ubiquitous promoter is selected from the group EFS promoter, CMV promoter and CAG promoter.

[13] A vector comprising a polynucleotide of any of [1] to [12].

[14] The vector of [13], wherein the vector is a plasmid vector or a viral vector.

[15] The vector of [14], wherein the viral vector is selected from the group adeno-associated virus (AAV) vector, adenovirus vector, and lentivirus vector.

[16] The vector of [15], wherein the AAV vector is selected from the group AAV1, AAV2, AAV6, AAV7, AAV8, AAV9, Anc80, AAV₅₈₇MTP, AAV₅₈₈MTP, AAV-B1, AAVM41, and AAVrh74.

[17] The vector of [16], wherein the AAV vector is AAV9.

[18] A pharmaceutical composition comprising a polynucleotide of any of [1] to [12] or a vector of any of [13] to [17].

[19] The pharmaceutical composition of [18] for treating or preventing Alzheimer's disease.

[20] A method for treating or preventing Alzheimer's disease, comprising administering a polynucleotide of any of [1] to [12], or a vector of any of [13] to [17], to a subject in need thereof.

### Advantageous Effects of Invention

According to the present invention, the expression of the human MAPT gene can be suppressed and, consequently, the present invention is expected to be able to treat tauopathy including AD.

A more complete appreciation of the invention and many of the attendant advantages thereof will be readily obtained as the same become better understood by reference to the following detailed description when considered in connection with the accompanying drawings.

### BRIEF DESCRIPTION OF DRAWINGS

FIG. 1 shows the relative sa sgRNA location to ‘UCSC Genome Browser on Human December 2013 (GRCh38/hg38) Assembly; chromosome 17: 45,887,381-45,962,898’.

FIG. 2 shows the results of evaluating the sa sgRNA for reducing MAPT mRNA levels between chromosome 17: 45,887,381-45,962,898 (UCSC Genome Browser on Human December 2013 (GRCh38/hg38) Assembly), within regions defined in FIG. 1.

FIG. 3 shows the results of evaluating the sa sgRNA efficacy for reducing MAPT mRNA levels between chromosome 17: 45,887,381-45,962,898 (UCSC Genome Browser on Human December 2013 (GRCh38/hg38) Assembly), within regions defined in FIG. 1.

### DESCRIPTION OF EMBODIMENTS

The embodiments of the present invention are explained in detail below.

1. Polynucleotide

The present invention provides a polynucleotide comprising the following base sequences (hereinafter sometimes to be also referred to as “the polynucleotide of the present invention”):

(a) a base sequence encoding a fusion protein of a nuclease-deficient CRISPR effector protein and a transcription repressor, and

(b) a base sequence encoding a guide RNA targeting a continuous region of 18 to 24 nucleotides (i.e., 18 to 24 contiguous nucleotides) in length in a region set forth in SEQ ID NO: 54, 55, 56, 57, 68 or 153, or 97 in the expression regulatory region of human MAPT gene. The region set forth in SEQ ID NO: 97 (CAGCTCCGGCACCAACAGCAGCGCCGCTGCCACCGCCCACCTTCTGCCGC CGCCACCACAGCCACCTTCTCCTCCTCCGCTGTCCTCTCCCGTCCTCGCCTC TGTCGACTATCAGGTAAGCGCCGCGGCTCCGAAATCTGCCTCGCCGTCCGC CTCTGTGCACCCCTGCGCCGCCGCCCCTCGCCCTCCCTCTCCGCAGACTGGG GCTTCGTGCGCCGGGCATCGGTCGGGGCCACCGCAGGGCCCCTCCCTGCCT CCCCTGCTCGGGGGCTGGGGCCAGGGCGGCCTGGAAAGGGACCTGAGCAA GGGATGCACGCACGC) comprises the regions set forth in SEQ ID NOs: 54, 55, 56 and 57.

The polynucleotide of the present invention is introduced into a desired cell and transcribed to produce a fusion protein of a nuclease-deficient CRISPR effector protein and a transcription repressor, and a guide RNA targeting a particular region of the expression regulatory region of the human MAPT gene. These fusion protein and guide RNA form a complex (hereinafter the complex is sometimes referred to as “ribonucleoprotein; RNP”) and cooperatively act on the aforementioned particular region, thus suppressing transcription of the human MAPT gene. In one embodiment of the present invention, the expression of the human MAPT gene can be suppressed by, for example, not less than about 40%, not less than about 50%, not less than about 60%, not less than about 70%, not less than about 75%, not less than about 80%, not less than about 85%, not less than about 90%, not less than about 95%, or about 100%.

### (1) Definition

In the present specification, “the expression regulatory region of human microtubule-associated protein tau (MAPT) gene” means any region in which the expression of human MAPT gene can be suppressed by binding RNP to that region. That is, the expression regulatory region of human MAPT gene may exist in any region such as the promoter region, enhancer region, intron, and exon of the human MAPT gene, as long as the expression of the human MAPT gene is suppressed by the binding of RNP. In the present specification, when the expression regulatory region is shown by the particular sequence, the expression regulatory region includes both the sense strand sequence and the antisense strand sequence conceptually.

In the present invention, a fusion protein of a nuclease-deficient CRISPR effector protein and a transcription repressor is recruited by a guide RNA into a particular region in the expression regulatory region of the human MAPT gene. In the present specification, the “guide RNA targeting . . . ” means a “guide RNA recruiting a fusion protein into . . . ”.

In the present specification, the “guide RNA (to be also referred to as ‘gRNA’)” is an RNA comprising a genome specific CRISPR-RNA (to be referred to as “crRNA”). crRNA is an RNA that binds to a complementary sequence of a targeting sequence (described later). When Cpf1 is used as the CRISPR effector protein, the “guide RNA” refers to an RNA comprising an RNA consisting of crRNA and a specific sequence attached to its 5′-terminal (for example, an RNA sequence set forth in SEQ ID NO: 101 in the case of FnCpf 1). When Cas9 is used as the CRISPR effector protein, the “guide RNA” refers to chimera RNA (to be referred to as “single guide RNA (sgRNA)”) comprising crRNA and trans-activating crRNA attached to its 3′-terminal (to be referred to as “tracrRNA”) (see, for example, Zhang F. et al., Hum Mol Genet. 2014 Sep. 15; 23(R1): R40-6 and Zetsche B. et al., Cell. 2015 Oct. 22; 163(3): 759-71, which are incorporated herein by reference in their entireties).

In the present specification, a sequence complementary to the sequence to which crRNA is bound in the expression regulatory region of the human MAPT gene is referred to as a “targeting sequence”. That is, in the present specification, the “targeting sequence” is a DNA sequence present in the expression regulatory region of the human MAPT gene and adjacent to PAM (protospacer adjacent motif). PAM is adjacent to the 5′-side of the targeting sequence when Cpf1 is used as the CRISPR effector protein. PAM is adjacent to the 3′-side of the targeting sequence when Cas9 is used as the CRISPR effector protein. The targeting sequence may be present on either the sense strand sequence side or the antisense strand sequence side of the expression regulatory region of the human MAPT gene (see, for example, the aforementioned Zhang F. et al., Hum Mol Genet. 2014 Sep. 15; 23(R1): R40-6 and Zetsche B. et al., Cell. 2015 Oct. 22; 163(3): 759-71, which are incorporated herein by reference in their entireties).

### (2) Nuclease-Deficient CRISPR Effector Protein

In the present invention, using a nuclease-deficient CRISPR effector protein, a transcriptional repressor fused thereto is recruited to the expression regulatory region of the human MAPT gene. The nuclease-deficient CRISPR effector protein (hereinafter to be simply referred to as “CRISPR effector protein”) to be used in the present invention is not particularly limited as long as it forms a complex with gRNA and is recruited to the expression regulatory region of the human MAPT gene. For example, nuclease-deficient Cas9 (hereinafter sometimes to be also referred to as “dCas9”) or nuclease-deficient Cpf1 (hereinafter sometimes to be also referred to as “dCpf1”) can be included.

Examples of the above-mentioned dCas9 include, but are not limited to, a nuclease-deficient variant of Streptococcus pyogenes-derived Cas9 (SpCas9; PAM sequence: NGG (N is A, G, T or C. hereinafter the same)), Streptococcus thermophilus-derived Cas9 (StCas9; PAM sequence: NNAGAAW (W is A or T. hereinafter the same)), Neisseria meningitidis-derived Cas9 (NmCas9; PAM sequence: NNNNGATT), or Staphylococcus aureus-derived Cas9 (SaCas9; PAM sequence: NNGRRT (R is A or G. hereinafter the same)) and the like (see, for example, Nishimasu et al., Cell. 2014 Feb. 27; 156(5): 935-49, Esvelt K M et al., Nat Methods. 2013 November; 10(11):1116-21, Zhang Y. Mol Cell. 2015 Oct. 15; 60(2):242-55, and Friedland A E et al., Genome Biol. 2015 Nov. 24; 16:257, which are incorporated herein by reference in their entireties). For example, in the case of SpCas9, a double mutant in which the 10th Asp residue is converted to Ala residue and the 840th His residue is converted to Ala residue (sometimes referred to as “dSpCas9”) can be used (see, for example, the aforementioned Nishimasu et al., Cell. 2014). Alternatively, in the case of SaCas9, a double mutant in which the 10th Asp residue is converted to Ala residue and the 580th Asn residue is converted to Ala residue (SEQ ID NO: 102), or a double mutant in which the 10th Asp residue is converted to Ala residue and the 557th His residue is converted to Ala residue (SEQ ID NO: 103) (hereinafter any of these double mutants is sometimes to be referred to as “dSaCas9”) can be used (see, for example, the aforementioned Friedland A E et al., Genome Biol. 2015, which is incorporated herein by reference in its entirety).

In addition, in one embodiment of the present invention, as dCas9, a variant obtained by modifying a part of the amino acid sequence of the aforementioned dCas9, which forms a complex with gRNA and is recruited to the expression regulatory region of the human MAPT gene, may also be used. Examples of such variants include a truncated variant with a partly deleted amino acid sequence. In one embodiment of the present invention, as dCas9, variants disclosed in WO2019/235627 and WO2020/085441, which are incorporated herein by reference in their entireties, can be used. Specifically, dSaCas9 obtained by deleting the 721st to 745th amino acids from dSaCas9 that is a double mutant in which the 10th Asp residue is converted to Ala residue and the 580th Asn residue is converted to Ala residue (SEQ ID NO: 104), or dSaCas9 in which the deleted part is substituted by a peptide linker (e.g., one in which the deleted part is substituted by GGSGGS linker (SEQ ID NO: 105) is set forth in SEQ ID NO: 106, and one in which the deleted part is substituted by SGGGS linker (SEQ ID NO: 107) is set forth in SEQ ID NO: 108, etc.) (hereinafter any of these double mutants is sometimes to be referred to as “dSaCas9[−25]”), or dSaCas9 obtained by deleting the 482nd to 648th amino acids from dSaCas9 that is the aforementioned double mutant (SEQ ID NO: 109), or dSaCas9 in which the deleted part is substituted by a peptide linker (one in which the deleted part is substituted by GGSGGS linker is set forth in SEQ ID NO: 110) may also be used.

Examples of the above-mentioned dCpf1 include, but are not limited to, a nuclease-deficient variant of Francisella novicida-derived Cpf1 (FnCpf1; PAM sequence: NTT), Acidaminococcus sp.-derived Cpf1 (AsCpf1; PAM sequence: NTTT), or Lachnospiraceae bacterium-derived Cpf1 (LbCpf1; PAM sequence: NTTT) and the like (see, for example, Zetsche B. et al., Cell. 2015 Oct. 22; 163(3):759-71, Yamano T et al., Cell. 2016 May 5; 165(4):949-62, and Yamano T et al., Mol Cell. 2017 Aug. 17; 67(4):633-45, which are incorporated herein by reference in their entireties). For example, in the case of FnCpf1, a double mutant in which the 917th Asp residue is converted to Ala residue and the 1006th Glu residue is converted to Ala residue can be used (see, for example, the aforementioned Zetsche B et al., Cell. 2015, which is incorporated herein by reference in its entirety). In one embodiment of the present invention, as dCpf1, a variant obtained by modifying a part of the amino acid sequence of the aforementioned dCpf1, which forms a complex with gRNA and is recruited to the expression regulatory region of the human MAPT gene, may also be used.

In one embodiment of the present invention, dCas9 is used as the nuclease-deficient CRISPR effector protein. In one embodiment, the dCas9 is dSaCas9, and, in a particular embodiment, the dSaCas9 is dSaCas9[−25].

A polynucleotide comprising a base sequence encoding a CRISPR effector protein can be cloned by, for example, synthesizing an oligoDNA primer covering a region encoding a desired part of the protein based on the cDNA sequence information thereof, and amplifying the polynucleotide by PCR method using total RNA or mRNA fraction prepared from the cells producing the protein as a template. In addition, a polynucleotide comprising a base sequence encoding a nuclease-deficient CRISPR effector protein can be obtained by introducing a mutation into a nucleotide sequence encoding a cloned CRISPR effector protein by a known site-directed mutagenesis method to convert the amino acid residues (e.g., 10th Asp residue, 557th His residue, and 580th Asn residue in the case of SaCas9; 917th Asp residue and 1006th Glu residue in the case of FnCpf1, and the like can be included, but are not limited to these) at a site important for DNA cleavage activity to other amino acids.

Alternatively, a polynucleotide comprising a base sequence encoding nuclease-deficient CRISPR effector protein can be obtained by chemical synthesis or a combination of chemical synthesis and PCR method or Gibson Assembly method, based on the cDNA sequence information thereof, and can also be further constructed as a base sequence that underwent codon optimization to give codons suitable for expression in human.

### (3) Transcriptional Repressor

In the present invention, human MAPT gene expression is repressed by the action of the transcriptional repressor fused with the nuclease-deficient CRISPR effector protein. In the present specification, the “transcriptional repressor” means a protein having the ability to repress gene transcription of human MAPT gene or a peptide fragment retaining the function thereof. The transcriptional repressor to be used in the present invention is not particularly limited as long as it can repress expression of human MAPT gene. It includes, for example, Kruppel-associated box (KRAB), MBD2B, vErbA, SID (including chain state of SID (SID4X)), MBD2, MBD3, DNMT family (e.g., DNMT1, DNMT3A, DNMT3B), Rb, MeCP2, ROM2, LSD1, AtHD2A, SET1, HDAC11, SETD8, EZH2, SUV39H1, PHF19, SALI, NUE, SUVR4, KYP, DIM5, HDAC8, SIRT3, SIRT6, MESOLO4, SET8, HST2, COBB, SET-TAF1B, NCOR, SIN3A, HDT1, NIPP1, HP1A, ERF repressor domain (ERD), and variants thereof having transcriptional repression ability, fusions thereof and the like. In one embodiment of the present invention, KRAB is used as the transcriptional repressor.

A polynucleotide comprising a base sequence encoding a transcriptional repressor can be constructed by chemical synthesis or a combination of chemical synthesis and PCR method or Gibson Assembly method. Furthermore, a polynucleotide comprising a base sequence encoding a transcriptional repressor can also be constructed as a codon-optimized DNA sequence to be codons suitable for expression in human.

A polynucleotide comprising a base sequence encoding a fusion protein of a transcriptional repressor and a nuclease-deficient CRISPR effector protein can be prepared by ligating a base sequence encoding the CRISPR effector protein to a base sequence encoding the transcriptional repressor directly or after adding a base sequence encoding a linker, NLS (nuclear localization signal)(for example, a base sequence set forth in SEQ ID NO: 111 or SEQ ID NO: 112), a tag and/or others. In the present invention, the transcriptional repressor may be fused with either N-terminal or C-terminal of the nuclease-deficient CRISPR effector protein. As the linker, a linker with an amino acid number of about 2 to 50 can be used, and specific examples thereof include, but are not limited to, a G-S-G-S linker in which glycine (G) and serine (S) are alternately linked and the like. In one embodiment of the present invention, as the polynucleotide comprising a base sequence encoding a fusion protein of a nuclease-deficient CRISPR effector protein and a transcriptional repressor, the base sequence set forth in SEQ ID NO: 113, which encodes SV40 NLS, dSaCas9, NLS and KRAB as a fused protein, can be used.

### (4) Guide RNA

In the present invention, a fusion protein of nuclease-deficient CRISPR effector protein and transcription repressor can be recruited to the expression regulatory region of the human MAPT gene by guide RNA. As described in the aforementioned “(1) Definition”, guide RNA comprises crRNA, and the crRNA binds to a complementary sequence of the targeting sequence. crRNA may not be completely complementary to the complementary sequence of the targeting sequence as long as the guide RNA can recruit the fusion protein to the target region, and may comprise a base sequence of the targeting sequence in which at least 1 to 3 bases are deleted, substituted, inserted and/or added.

When dCas9 is used as the nuclease-deficient CRISPR effector protein, for example, the targeting sequence can be determined using a published gRNA design web site (CRISPR Design Tool, CRISPR direct, etc.). To be specific, from the sequence of the target gene (i.e., human MAPT gene), candidate targeting sequences of about 20 nucleotides in length for which PAM (e.g., NNGRRT in the case of SaCas9) is adjacent to the 3′-side thereof are listed, and one having a small number of off-target sites in human genome from among these candidate targeting sequences can be used as the targeting sequence. The base length of the targeting sequence is 18 to 24 nucleotides in length, preferably 20 to 23 nucleotides in length, more preferably 21 to 23 nucleotides in length. As a primary screening for the prediction of the off-target site number, a number of bioinformatic tools are known and publicly available, and can be used to predict the targeting sequence with the lowest off-target effect. Examples thereof include bioinformatics tools such as Benchling (https://benchling.com), and COSMID (CRISPR Off-target Sites with Mismatches, Insertions, and Deletions) (Available on https://crispr.bme.gatech.edu on the internet). Using these, the similarity to the base sequence targeted by gRNA can be summarized. When the gRNA design software to be used does not have a function to search for off-target site of the target genome, for example, the off-target site can be searched for by subjecting the target genome to Blast search with respect to 8 to 12 nucleotides on the 3′-side of the candidate targeting sequence (seed sequence with high discrimination ability of targeted nucleotide sequence).

In one embodiment of the present invention, in the region existing in the GRCh38/hg38 of human chromosome 17 (Chr 17), the region of “45,887,381-45,962,898” can be the expression regulatory region of the human MAPT gene. Therefore, in one embodiment of the present invention, the targeting sequence can be 18 to 24 nucleotides in length, preferably 20 to 23 nucleotides in length, more preferably 21 to 23 nucleotides in length, in the regions of “45,887,381-45,962,898” existing in the GRCh38/hg38 of human chromosome 17 (Chr 17).

In one embodiment of the present invention, a base sequence encoding crRNA may be the same base sequence as the targeting sequence. For example, when the targeting sequence set forth in SEQ ID NO: 57 (GAGCAAGGGATGCACGCACG) is introduced into the cell as a base sequence encoding crRNA, crRNA transcribed from the sequence is GAGCAAGGGAUGCACGCACG (SEQ ID NO:114) and is bound to CGTGCGTGCATCCCTTGCTC (SEQ ID NO: 115), which is a sequence complementary to the base sequence set forth in SEQ ID NO: 57 and is present in the expression regulatory region of the human MAPT gene. In another embodiment, a base sequence which is a targeting sequence in which at least 1 to 3 bases are deleted, substituted, inserted and/or added can be used as the base sequence encoding crRNA as long as guide RNA can recruit a fusion protein to the target region. Therefore, in one embodiment of the present invention, as a base sequence encoding crRNA, the base sequence set forth in SEQ ID NO: 54, 55, 56, 57, 68, 153 or 97, or the base sequence set forth in SEQ ID NO: 54, 55, 56, 57, 68, 153 or 97 in which 1 to 3 bases are deleted, substituted, inserted and/or added can be used.

When dCpf1 is used as the nuclease-deficient CRISPR effector protein, a base sequence encoding gRNA can be designed as a DNA sequence encoding crRNA with particular RNA attached to the 5′-terminal. Such RNA attached to the 5′-terminal of crRNA and a DNA sequence encoding said RNA can be appropriately selected by those of ordinary skill in the art according to the dCpf1 to be used. For example, when dFnCpf1 is used, a base sequence in which SEQ ID NO:116; AATTTCTACTGTT GTAGAT is attached to the 5′-side of the targeting sequence can be used as a base sequence encoding gRNA (when transcribed to RNA, the sequences of the underlined parts form base pairs to form a stem-loop structure). The sequence to be added to the 5′-terminal may be a sequence generally used for various Cpf1 proteins in which at least 1 to 6 bases are deleted, substituted, inserted and/or added, as long as gRNA can recruit a fusion protein to the expression regulatory region after transcription.

When dCas9 is used as the CRISPR effector protein, a base sequence encoding gRNA can be designed as a DNA sequence in which a DNA sequence encoding known tracrRNA is linked to the 3′-terminal of a DNA sequence encoding crRNA. Such tracrRNA and a DNA sequence encoding the tracrRNA can be appropriately selected by those of ordinary skill in the art according to the dCas9 to be used. For example, when dSaCas9 is used, the base sequence set forth in SEQ ID NO: 117 is used as the DNA sequence encoding tracrRNA. The DNA sequence encoding tracrRNA may be a base sequence encoding tracrRNA generally used for various Cas9 proteins in which at least 1 to 6 bases are deleted, substituted, inserted and/or added, as long as gRNA can recruit a fusion protein to the expression regulatory region after transcription.

A polynucleotide comprising a base sequence encoding gRNA designed in this way can be chemically synthesized using a known DNA synthesis method.

In another embodiment of the present invention, the polynucleotide of the present invention may comprise at least two different base sequences encoding a gRNA. For example, the polynucleotide can comprise at least two different base sequences encoding the guide RNA, wherein the at least two different base sequences are selected from a base sequence comprising a sequence set forth in SEQ ID NO: 54, 55, 56, 57, 68, 153 or 97.

### (5) Promoter Sequence

In one embodiment of the present invention, a promoter sequence may be operably linked to the upstream of each of a base sequence encoding fusion protein of nuclease-deficient CRISPR effector protein and transcriptional repressor and/or a base sequence encoding gRNA. The promoter to be possibly linked is not particularly limited as long as it shows a promoter activity in the target cell. Examples of the promoter sequence possibly linked to the upstream of the base sequence encoding gRNA include, but are not limited to, U6 promoter, SNR6 promoter, SNR52 promoter, SCR1 promoter, RPR1 promoter, U3 promoter, H1 promoter, and tRNA promoter, which are pol III promoters, and the like. In one embodiment of the present invention, U6 promoter can be used as the promoter sequence for the base sequence encoding the guide RNA. In one embodiment of the present invention, when a polynucleotide comprises two or more base sequences respectively encoding a guide RNA, a single promoter sequence may be operably linked to the upstream of the two or more base sequences. In another embodiment, when a polynucleotide comprises two or more base sequences respectively encoding a guide RNA, a promoter sequence may be operably linked to the upstream of each of the two or more base sequences, wherein the promoter sequence operably linked to each base sequence may be the same or different.

As the aforementioned promoter sequence possibly linked to the upstream of the base sequence encoding fusion protein, a ubiquitous promoter or neuron-specific promoter may be used. Examples of the ubiquitous promoter include, but are not limited to, EF-1a promoter, EFS promoter, CMV (cytomegalovirus) promoter, hTERT promoter, SRa promoter, SV40 promoter, LTR promoter, CAG promoter, RSV (Rous sarcoma virus) promoter, and the like. In one embodiment of the present invention, EFS promoter, CMV promoter or CAG promoter can be used as the ubiquitous promoter. Examples of the neuron-specific promoter include, but are not limited to, neuron-specific enolase (NSE) promoter, human neurofilament light chain (NEFL) promoter. The aforementioned promoter may have any modification and/or alteration as long as it has promoter activity in the target cell.

In one embodiment of the present invention, U6 is used as a promoter for a base sequence encoding the guide RNA, and CMV promoter can be used as the promoter sequence for the base sequence encoding the fusion protein.

### (6) Other Base Sequence

Furthermore, the polynucleotide of the present invention may further comprise known sequences such as polyadenylation (polyA) signal, Kozak consensus sequence and the like besides those mentioned above for the purpose of improving the translation efficiency of mRNA produced by transcription of a base sequence encoding a fusion protein of nuclease-deficient CRISPR effector protein and transcription repressor. For example, polyadenylation signal in the present invention may include hGH polyA, bGH polyA, 2×sNRP-1 polyA (see U.S. Pat. No. 7,557,197B2, which is incorporated herein by reference in its entirety), and so on. In addition, the polynucleotide of the present invention may comprise a base sequence encoding a linker sequence, a base sequence encoding NLS and/or a base sequence encoding a tag. Furthermore, the polynucleotide of the present invention may comprise an intervening sequence. A preferred example of the intervening sequence is a sequence encoding IRES (Internal ribosome entry site), 2A peptide. The 2A peptide is a peptide sequence of around 20 amino acid residues derived from virus, is recognized by a protease present in the cell (2A peptidase), and is cleaved at the position of 1 residue from the C terminal. Multiple genes linked as one unit by 2A peptide are transcribed and translated as one unit, and then cleaved by 2A peptidase. Examples of the 2A peptidase include F2A (derived from foot-and-mouth disease virus), E2A (derived from equine rhinitis A virus), T2A (derived from Thosea asigna virus), and P2A (derived from porcine teschovirus-1).

### (7) Exemplified Embodiments of the Present Invention

In one embodiment of the present invention, a polynucleotide is provided comprising:

a base sequence encoding a fusion protein of a nuclease-deficient CRISPR effector protein and a transcriptional repressor,

a promoter sequence for the base sequence encoding the fusion protein of the nuclease-deficient CRISPR effector protein and the transcriptional repressor,

one or two base sequences respectively encoding a guide RNA, wherein the one or two base sequences are selected from a base sequence comprising a sequence set forth in SEQ ID NO: 54, 55, 56, 57, 68, 153 or 97, or the base sequence comprising a sequence set forth in SEQ ID NO: 54, 55, 56, 57, 68, 153 or 97, in which 1 to 3 bases are deleted, substituted, inserted, and/or added, and

a promoter sequence for the base sequence encoding the gRNA,

wherein the nuclease-deficient CRISPR effector protein is dSaCas9 or dSaCas9[−25],

wherein the transcriptional repressor is selected from the group KRAB, MeCP2, SIN3A, HDT1, MBD2B, NIPP1, and HP1A,

wherein the promoter sequence for the base sequence encoding the fusion protein is selected from the group EFS promoter, CMV promoter and CAG promoter, and

wherein the promoter sequence for the base sequence encoding the gRNA is selected from the group U6 promoter, SNR6 promoter, SNR52 promoter, SCR1 promoter, RPR1 promoter, U3 promoter, and H1 promoter.

In one embodiment of the present invention, a polynucleotide is provided comprising:

a base sequence encoding a fusion protein of a nuclease-deficient CRISPR effector protein and a transcriptional repressor,

CMV promoter for the base sequence encoding the fusion protein of the nuclease-deficient CRISPR effector protein and the transcriptional repressor,

one or two base sequences respectively encoding a guide RNA, wherein the one or two base sequences are selected from a base sequence comprising a sequence set forth in SEQ ID NO: 54, 55, 56, 57, 68, 153 or 97, or a base sequence comprising a sequence set forth in SEQ ID NO: 54, 55, 56, 57, 68, 153 or 97 in which 1 to 3 bases are deleted, substituted, inserted, and/or added, and U6 promoter for the base sequence encoding the guide RNA,

wherein the nuclease-deficient CRISPR effector protein is dSaCas9, and
wherein the transcriptional repressor is KRAB.

2. Vector

The present invention provides a vector comprising the polynucleotide of the present invention (hereinafter sometimes referred to as “the vector of the present invention”). The vector of the present invention may be a plasmid vector or a viral vector.

When the vector of the present invention is a plasmid vector, the plasmid vector to be used is not particularly limited and may be any plasmid vector such as cloning plasmid vector and expression plasmid vector. The plasmid vector is prepared by inserting the polynucleotide of the present invention into a plasmid vector by a known method.

When the vector of the present invention is a viral vector, the viral vector to be used is not particularly limited and examples thereof include, but are not limited to, adenovirus vector, adeno-associated virus (AAV) vector, lentivirus vector, retrovirus vector, Sendaivirus vector and the like. In the present specification, the “virus vector” or “viral vector” also includes derivatives thereof. Considering the use in gene therapy, AAV vector is preferably used for the reasons such that it can express transgene for a long time, and it is derived from a non-pathogenic virus and has high safety.

A viral vector comprising the polynucleotide of the present invention can be prepared by a known method. In brief, a plasmid vector for virus expression into which the polynucleotide of the present invention has been inserted is prepared, the vector is transfected into an appropriate host cell to allow for transient production of a viral vector comprising the polynucleotide of the present invention, and the viral vector is collected.

In one embodiment of the present invention, when AAV vector is used, the serotype of the AAV vector is not particularly limited as long as expression of the human MAPT gene in the target can be activated, and any of AAV1, AAV2, AAV3, AAV4, AAV5, AAV6, AAV7, AAV8, AAV9, AAVrh.10 and the like may be used (for the various serotypes of AAV, see, for example, WO 2005/033321 and EP2341068 (A1), which are incorporated herein by reference in their entireties). Examples of the variants of AAV include, but are not limited to, new serotype with a modified capsid (e.g., WO 2012/057363, which is incorporated herein by reference in its entirety) and the like. For example, in one embodiment of the present invention, a new serotype with a modified capsid improving infectivity for muscle cells can be used, such as AAV₅₈₇ MTP, AAV₅₈₈MTP, AAV-B1, AAVM41, AAVS1_P1, and AAVS10_P1, and the like (see Yu et al., Gene Ther. 2009 August; 16(8):953-62, Choudhury et al., Mol Ther. 2016 August; 24(7):1247-57, Yang et al., Proc Natl Acad Sci USA. 2009 Mar. 10; 106(10):3946-51, and WO2019/207132, which are incorporated herein by reference in their entireties).

When an AAV vector is prepared, a known method such as (1) a method using a plasmid, (2) a method using a baculovirus, (3) a method using a herpes simplex virus, (4) a method using an adenovirus, or (5) a method using yeast can be used (e.g., Appl Microbiol Biotechnol. 2018; 102(3): 1045-1054, etc., which is incorporated herein by reference in its entirety). For example, when an AAV vector is prepared by a method using a plasmid, first, a vector plasmid comprising inverted terminal repeat (ITR) at both ends of wild-type AAV genomic sequence and the polynucleotide of the present invention inserted in place of the DNA encoding Rep protein and capsid protein is prepared. On the other hand, the DNA encoding Rep protein and capsid protein necessary for forming virus particles are inserted into other plasmids. Furthermore, a plasmid comprising genes (E1A, E1B, E2A, VA and E4orf6) responsible for the helper action of adenovirus necessary for proliferation of AAV is prepared as an adenovirus helper plasmid. The co-transfection of these three kinds of plasmids into the host cell causes the production of recombinant AAV (i.e., AAV vector) in the cell. As the host cell, a cell capable of supplying a part of the gene products (proteins) of the genes responsible for the aforementioned helper action (e.g., 293 cell, etc.) is preferably used. When such cell is used, it is not necessary to carry the gene encoding a protein that can be supplied from the host cell in the aforementioned adenoviral helper plasmid. The produced AAV vector is present in the nucleus. Thus, a desired AAV vector is prepared by destroying the host cell with freeze-thawing, collecting the virus and then subjecting the virus fraction to separation and purification by density gradient ultracentrifugation method using cesium chloride, column method or the like.

AAV vector has great advantages in terms of safety, gene transduction efficiency and the like, and is used for gene therapy. However, it is known that the size of a polynucleotide that can be packaged in AAV vector is limited. For example, in one embodiment of the present invention, the entire length including the base length of a polynucleotide comprising a base sequence encoding a fusion protein of dSaCas9 and miniVR or microVR, a base sequence encoding gRNA targeting the expression regulatory region of the human MAPT gene, and EFS promoter sequence or CK8 promoter sequence and U6 promoter sequence as the promoter sequences, and ITR parts is about 4.85 kb, and they can be packaged in a single AAV vector.

3. Pharmaceutical Composition

The present invention also provides a pharmaceutical composition comprising the polynucleotide of the present invention or the vector of the present invention (hereinafter sometimes referred to as “the pharmaceutical composition of the present invention”). The pharmaceutical composition of the present invention can be used for treating or preventing tauopathy including AD.

The pharmaceutical composition of the present invention comprises the polynucleotide of the present invention or the vector of the present invention as an active ingredient, and may be prepared as a formulation comprising such active ingredient (i.e., the polynucleotide of the present invention or the vector of the present invention) and, generally, a pharmaceutically acceptable carrier.

The pharmaceutical composition of the present invention is administered parenterally, and may be administered topically or systemically. The pharmaceutical composition of the present invention can be administered by, but are not limited to, for example, intravenous administration, intraarterial administration, subcutaneous administration, intraperitoneal administration, or intramuscular administration.

The dose of the pharmaceutical composition of the present invention to a subject is not particularly limited as long as it is an effective amount for the treatment and/or prevention. It may be appropriately optimized according to the active ingredient, dosage form, age and body weight of the subject, administration schedule, administration method and the like.

In one embodiment of the present invention, the pharmaceutical composition of the present invention can be not only administered to the subject affected with tauopathy including AD but also prophylactically administered to subjects who may develop tauopathy including AD in the future based on the genetic background analysis and the like. The term “treatment” in the present specification also includes remission of disease, in addition to the cure of diseases. In addition, the term “prevention” may also include delaying the onset of disease, in addition to prophylaxis of the onset of disease. The pharmaceutical composition of the present invention can also be referred to as “the agent of the present invention” or the like.

4. Method for Treatment or Prevention of DMD or BMD

The present invention also provides a method for treating or preventing tauopathy including AD, comprising administering the polynucleotide of the present invention or the vector of the present invention to a subject in need thereof (hereinafter sometimes referred to as “the method of the present invention”). In addition, the present invention includes the polynucleotide of the present invention or the vector of the present invention for use in the treatment or prevention of tauopathy including AD. Furthermore, the present invention includes use of the polynucleotide of the present invention or the vector of the present invention in the manufacture of a pharmaceutical composition for the treatment or prevention of tauopathy including AD.

The method of the present invention can be practiced by administering the aforementioned pharmaceutical composition of the present invention to a subject affected with tauopathy including AD, and the dose, administration route, subject and the like are the same as those mentioned above.

Measurement of the symptoms may be performed before the start of the treatment using the method of the present invention and at any timing after the treatment to determine the response of the subject to the treatment.

The method of the present invention can improve the functions of the skeletal muscle and/or cardiac muscle of the subject. Muscles to be improved in the function thereof are not particularly limited, and any muscles and muscle groups are exemplified.

5. Ribonucleoprotein

The present invention provides a ribonucleoprotein comprising the following (hereinafter sometimes referred to as “RNP of the present invention”):

(c) a fusion protein of a nuclease-deficient CRISPR effector protein and a transcription repressor, and

(d) a guide RNA targeting a continuous region of 18 to 24 nucleotides in length in a region set forth in SEQ ID NO: 54, 55, 56, 57, 68, 153 or 97 in the expression regulatory region of human MAPT gene.

As the nuclease-deficient CRISPR effector protein, transcription repressor, and guide RNA comprised in the RNP of the present invention, the nuclease-deficient CRISPR effector protein, transcription repressor, and guide RNA explained in detail in the above-mentioned section of “1. Polynucleotide” can be used. The fusion protein of nuclease-deficient CRISPR effector protein and transcription repressor to be comprised in the RNP of the present invention can be produced by, for example, introducing a polynucleotide encoding the fusion protein into the cell, bacterium, or other organism to allow for the expression, or an in vitro translation system by using the polynucleotide. In addition, guide RNA comprised in the RNP of the present invention can be produced by, for example, chemical synthesis or an in vitro transcription system by using a polynucleotide encoding the guide RNA. The thus-prepared fusion protein and guide RNA are mixed to prepare the RNP of the present invention. Where necessary, other substances such as gold particles may be mixed. To directly deliver the RNP of the present invention to the target cell, tissue and the like, the RNP may be encapsulated in a lipid nanoparticle (LNP) by a known method. The RNP of the present invention can be introduced into the target cell, tissue and the like by a known method. For example, Lee K., et al., Nat Biomed Eng. 2017; 1:889-901, WO 2016/153012, which are incorporated herein by reference in their entireties, and the like can be referred to for encapsulation in LNP and introduction method.

In one embodiment of the present invention, the guide RNA comprised in RNP of the present invention targets continuous 18 to 24 nucleotides in length, preferably 20 to 23 nucleotides in length, more preferably 21 to 23 nucleotides in length, in the region of “45,887,381-45,962,898” existing in the GRCh38/hg38 of human chromosome 17 (Chr 17).

6. Others

The present invention also provides a composition or kit comprising the following for suppression of the expression of the human MAPT gene:

(e) a fusion protein of a nuclease-deficient CRISPR effector protein and a transcription repressor, or a polynucleotide encoding the fusion protein, and

(f) a guide RNA targeting a continuous region of 18 to 24 nucleotides in length in a region set forth in SEQ ID NO: 54, 55, 56, 57, 68, 153 or 97 in the expression regulatory region of human MAPT gene, or a polynucleotide encoding the guide RNA.

The present invention also provides a method for treating or preventing tauopathy including AD, comprising administering the following (e) and (f):

(e) a fusion protein of a nuclease-deficient CRISPR effector protein and a transcription repressor, or a polynucleotide encoding the fusion protein, and

(f) a guide RNA targeting a continuous region of 18 to 24 nucleotides in length in a region set forth in SEQ ID NO: 54, 55, 56, 57, 68, 153 or 97 in the expression regulatory region of human MAPT, or a polynucleotide encoding the guide RNA.

The present invention also provides use of the following (e) and (f):

(e) a fusion protein of a nuclease-deficient CRISPR effector protein and a transcription repressor, or a polynucleotide encoding the fusion protein, and

(f) a guide RNA targeting a continuous region of 18 to 24 nucleotides in length in a region set forth in SEQ ID NO: 54, 55, 56, 57, 68, 153 or 97 in the expression regulatory region of human MAPT gene, or a polynucleotide encoding the guide RNA, in the manufacture of a pharmaceutical composition for the treatment or prevention of tauopathy including AD.

As the nuclease-deficient CRISPR effector protein, transcription repressor, guide RNA, as well as polynucleotides encoding them and vectors in which they are carried in these inventions, those explained in detail in the above-mentioned sections of “1. Polynucleotide”, “2. Vector” and “5. Ribonucleoprotein” can be used. The dose, administration route, subject, formulation and the like of the above-mentioned (e) and (f) are the same as those explained in the section of “3. Pharmaceutical composition”.

Other features of the invention will become apparent in the course of the following descriptions of exemplary embodiments which are given for illustration of the invention and are not intended to be limiting thereof.

### EXAMPLES

The examples describe the use of a fusion protein of dCas9 with a transcriptional repressor to suppress gene expression, in the defined expression regulatory region of human MAPT gene that leads to the selective suppression of human MAPT gene expression. The example also describes the definition of a specific genomic region that confers selective suppression of the human MAPT gene without minimally affecting the expression of other genes. The method of the present invention to suppress human MAPT gene expression represents a novel therapeutic or preventive strategy for the tauopathy including AD as described and illustrated herein.

### Example 1

(1) Experimental Methods

Cell Culture and Transfection

SK-N-AS (American Type Culture Collection) cells were seeded 24 hours prior to transfection in 12-well plates at a density of 100,000 cells per well and cultured in DMEM media supplemented with 10% FBS and 2 mM fresh L-glutamine, 1 mM sodium pyruvate and non-essential amino acids. Cells were transfected with 1000 ng sgRNA containing px601-CMV-dSaCas9-KRAB-P2A-Puro (modified from GenScript) plasmid using 3.0 μl of TransIT-VirusGEN (Mirus Bio), according to manufacturer's instructions.

For gene expression analysis, the transfected cells were harvested at 72 h after transfection and lysed in RLT buffer (Qiagen) to extract total RNA using RNeasy kit (Qiagen).

Gene Expression Analysis

For Taqman analysis, 1.5 μg of total RNA was used to generate cDNA using TaqMan High-Capacity RNA-to-cDNA Kit (Applied Biosystems) in 20 μl volume. The generated cDNA was diluted 10-fold and 3.33 μl was used per Taqman reaction. The Taqman primers and probes for the MAPT and HPRT gene were obtained from Applied Biosystems. Taqman reaction was run using Taqman gene expression master mix (ThermoFisher) in ThermoFisher QuantStudio 5 Real-Time PCR System and analyzed using QuantStudio 5 analysis software.

Taqman probe product IDs:

MAPT: Hs00902193_ml (FAM-MGB)

HPRT: Hs99999909_ml (VIC PL)

Taqman qPCR condition:

Step 1; 50° C. 2 min

Step 2; 95° C. 2 min

Step 3; 95° C. 1 sec

Step 4; 60° C. 20 sec

Repeat Steps 3 and 4; 45 times

Selection of sgRNA Sequence

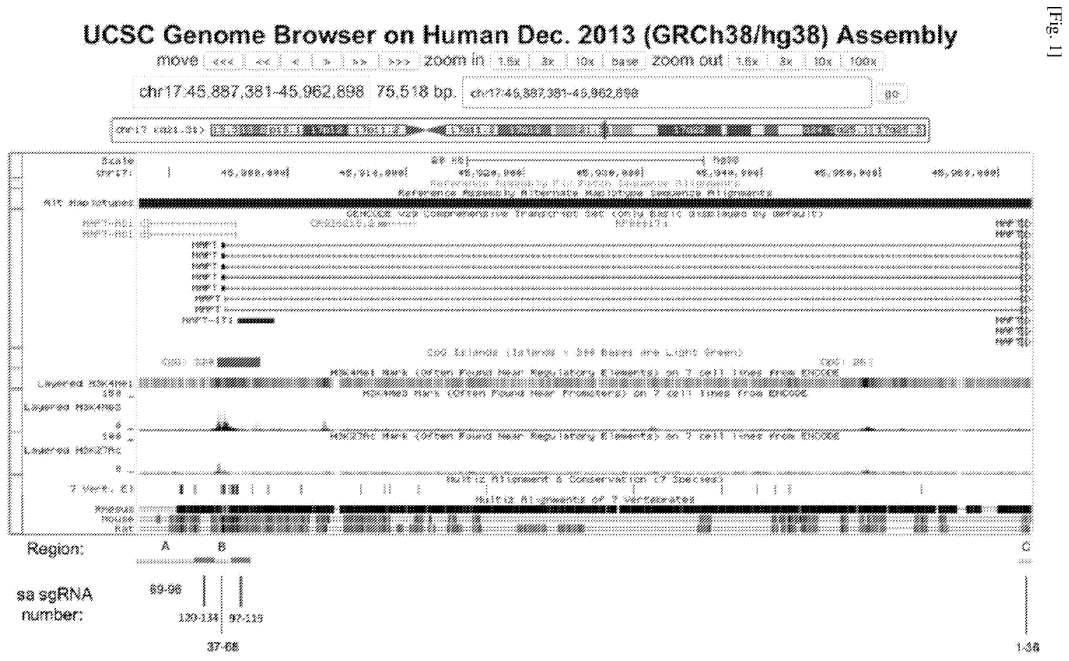

The location of the guide RNA target sites relative to the MAPT gene is shown in FIG. 1. The selected guide RNA sequences (Table 1) or control sgRNA guide sequences (Table 2) were fused with the tracer RNA sequence to form single-molecule guide RNA (sgRNA) sequences, and were cloned into

px601-CMV-dSaCas9-KRAB-P2A-Puro (modified from GenScript). The sgRNA expression is driven by the hU6 promoter, and the vector expresses the puromycin gene under a CMV/P2A promoter to facilitate tracking and selection of the sgRNA expressing cells. Three control sgRNA guides (Table 2) were selected from the Human CRISPR Knockout Pooled Library (Sanjana N. E. et al, Nature Methods, 11(8), p. 783, 2014).

(2) Results

Suppression of MAPT Gene Expression by the RNP

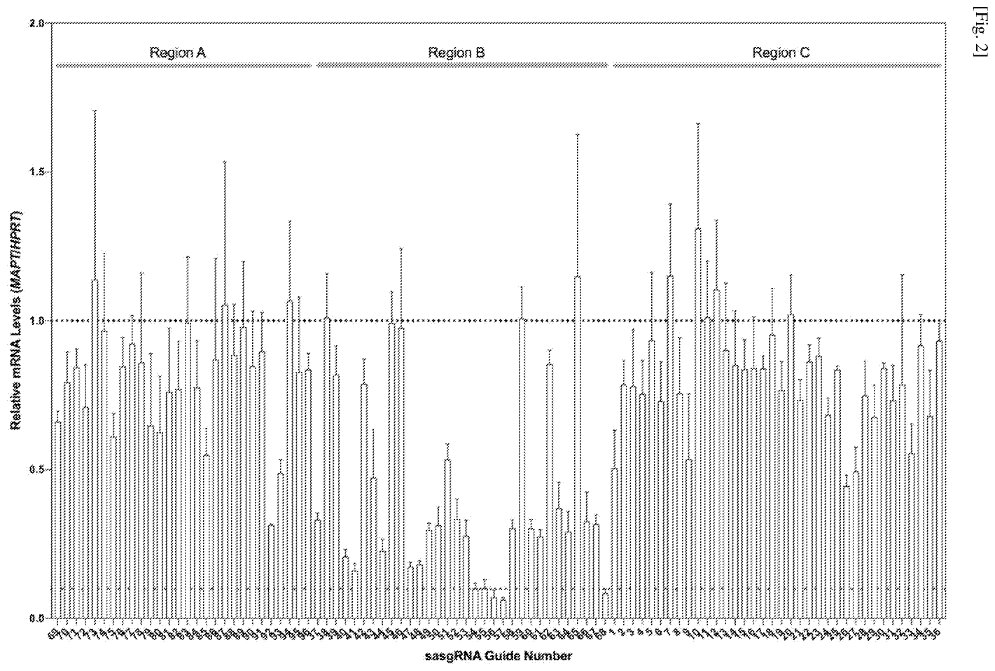

The suppression of MAPT transcript by the ninety-six sgRNAs are shown (FIG. 2), where MAPT transcript expression was normalized to the mRNA levels of the HPRT gene. Detected MAPT expression levels after transfection with control sgRNA guides were set to 1.0. sgRNA #54, 55, 56, 57, and 68 showed ≥90% suppression (FIG. 2). The experiments detailed were conducted at least three times, and the mean-fold suppression values and standard deviations are shown.

Table 1 List of sa sgRNA guides (with NNGRRT PAM sequence) within ‘UCSC Genome Browser on Human December 2013 (GRCh38/hg38) Assembly; chromosome 17: 45,887,381-45,962,898’.

TABLE 1-1

| SEQ ID | Guide RNA | | | | | Specificity | Efficiency |
|---|---|---|---|---|---|---|---|
| No. | (sa sgRNA) | Position | Strand | Sequence | PAM | Score | Score |
| 1 | 1 | 45961816 | -1 | ACTGCACTCCAGCCTGGGTGA | CAGGGT | 4.2707957 | 11.1054033 |
| 2 | 2 | 45961820 | 1 | GTTTCACCCTGTCACCCAGGC | TTGAGT | 35.0472918 | 7.38177467 |
| 3 | 3 | 45961824 | -1 | ATTACACCACTGCACTCCAGC | CTGGGT | 19.503033 | 54.5412524 |
| 4 | 4 | 45961889 | -1 | CTACTGGGGAGGCTGAGGCAG | GAGAAT | 13.1602659 | 14.4495198 |
| 5 | 5 | 45961905 | 1 | TGCCTCAGCCTCCCCAGTAGC | TGGGAT | 48.3443421 | 5.93261184 |
| 6 | 6 | 45961960 | 1 | TTATTGTATTTTTAGTAGAGA | TGGGGT | 7.2384615 | 1.89303844 |
| 7 | 7 | 45962005 | -1 | TGGGTGGATCACCTGAGGTCA | GGGGAT | 77.1434169 | 6.39485571 |
| 8 | 8 | 45962023 | -1 | CACTTTGGGAGGCCGAGGTGG | GTGGAT | 39.4034708 | 18.9296388 |
| 9 | 9 | 45962027 | -1 | TCAGCACTTTGGGAGGCCGAG | GTGGGT | 31.821238 | 31.2362459 |
| 10 | 10 | 45962086 | 1 | CCTGGCCGTCACCTGGTGGTG | TTGAAT | 75.6281371 | 1.77814841 |
| 11 | 11 | 45962151 | 1 | AGATGTAAATAACGCTTGGGC | AGGAAT | 94.3928814 | 9.98943822 |
| 12 | 12 | 45962165 | 1 | CTTGGGCAGGAATATGGAGCA | CGGGAT | 69.680597 | 3.89982682 |
| 13 | 13 | 45962171 | 1 | CAGGAATATGGAGCACGGGAT | GAGGAT | 87.4269228 | 11.1391751 |
| 14 | 14 | 45962195 | 1 | GATGGGCGGCCAACTGTTAGA | GAGGGT | 94.4779443 | 43.0454744 |
| 15 | 15 | 45962272 | -1 | AGGAGTGTTGGGGGGCAGAGT | GGGGGT | 46.3644966 | 5.21116914 |
| 16 | 16 | 45962278 | -1 | GTTCTGAGGAGTGTTGGGGGG | CAGAGT | 53.512248 | 15.1839364 |
| 17 | 17 | 45962293 | -1 | AAGAGGAGAGGATAAGTTCTG | AGGAGT | 69.2107764 | 35.7827944 |
| 18 | 18 | 45962307 | -1 | TCACCTGGGGAAAGAAGAGGA | GAGGAT | 39.5320546 | 5.00525373 |
| 19 | 19 | 45962331 | 1 | TTCCCCAGGTGAACTTTGAAC | CAGGAT | 77.6067082 | 25.3739037 |
| 20 | 20 | 45962351 | 1 | CCAGGATGGCTGAGCCCCGCC | AGGAGT | 74.313096 | 8.1697096 |
| 21 | 21 | 45962387 | 1 | TGGAAGATCACGCTGGGACGT | ACGGGT | 92.4905661 | 3.51725961 |
| 22 | 22 | 45962440 | 1 | TACACCATGCACCAAGACCAA | GAGGGT | 81.5779043 | 63.9977136 |
| 23 | 23 | 45962513 | 1 | GGCCCAGATCACTGCAAGCCA | AGGGGT | 78.3689171 | 22.4204299 |
| 24 | 24 | 45962538 | 1 | GTGGCGGGAACAGTTTGCATC | CAGAAT | 80.3998337 | 7.57883596 |
| 25 | 25 | 45962547 | -1 | ATTTAAAATTTCTTTGCAATT | CTGGAT | 44.678718 | 1.35233247 |
| 26 | 26 | 45962605 | 1 | GTAAAGTAAAGCCTCATTAAT | TTGAGT | 55.7696345 | 2.75396296 |
| 27 | 27 | 45962634 | -1 | GTCTGGCCATTATCTCACTGC | TTGAGT | 76.3653348 | 4.70638165 |
| 28 | 28 | 45962680 | -1 | TGCCTGGGCCTTCCAAAGTGC | TGGGAT | 26.7935865 | 13.2396766 |
| 29 | 29 | 45962696 | 1 | CACTTTGGAAGGCCCAGGCAG | GAGGAT | 34.83137 | 30.2924901 |
| 30 | 30 | 45962712 | -1 | GGTCTCAAATTCCTGGCCTCA | AGGGAT | 76.867643 | 1.73623484 |
| 31 | 31 | 45962713 | 1 | GCAGGAGGATCCCTTGAGGCC | AGGAAT | 25.7962436 | 4.81698985 |
| 32 | 32 | 45962759 | -1 | ATTTTTAAATTATTTTAGAGA | CGGGGT | 33.8490644 | 7.01995763 |
| 33 | 33 | 45962814 | 1 | ATGTCTATAGTCCTAGCTACT | CAGGAT | 73.1152589 | 13.3784542 |
| 34 | 34 | 45962823 | -1 | TGATCCTTCTGCCTCAGCATC | CTGAGT | 70.752644 | 12.3488214 |
| 35 | 35 | 45962830 | 1 | CTACTCAGGATGCTGAGGCAG | AAGGAT | 26.6698191 | 9.87147671 |
| 36 | 36 | 45962847 | 1 | GCAGAAGGATCACTTGAGCCC | AGGAGT | 57.4490484 | 22.7859368 |
| 33 | 37 | 45893571 | -1 | AGTGGGATGATTTCTATGTAG | GGGGGT | 77.3291451 | 16.9076608 |
| 38 | 38 | 45893590 | -1 | ATCAAGTTTAAGCCCAAGCAG | TGGGAT | 78.0130617 | 67.2473985 |
| 39 | 39 | 45893633 | -1 | GCCCCAGGCTTCGGCCTTAGC | TTGGAT | 84.965362 | 2.0071445 |
| 40 | 40 | 45893660 | 1 | GCCTGGGGCCTGGGCAGACAG | CAGAAT | 74.2151504 | 32.6101525 |
| 41 | 41 | 45893746 | -1 | GCTGTAAATAGAGCTTGAAGT | CTGAAT | 65.5490417 | 60.3636159 |
| 42 | 42 | 45893843 | -1 | GAAAAAAAAATCTTAAATTAG | ACGAAT | 38.227743 | 4.28568603 |
| 43 | 43 | 45893885 | -1 | TGTACTTAAAAAAAAAGAAAC | GTGAAT | 39.0517239 | 37.3185904 |
| 44 | 44 | 45893917 | 1 | TACAGTTCTACTGTATTGTAA | CTGAGT | 73.9591349 | 57.6998631 |
| 45 | 45 | 45893952 | 1 | TTTAAGCCGATTTGTTAAGGA | AAGGAT | 89.1307684 | 23.8637812 |
| 46 | 46 | 45893968 | -1 | CCTTTTTTGTTACTGACCAAG | GTGAAT | 72.020046 | 29.020082 |
| 47 | 47 | 45894058 | 1 | GGGGGGCGGTTTCGGACTACG | AAGGGT | 98.364443 | 16.0031071 |
| 48 | 48 | 45894088 | -1 | GGCCTTCCACGTGGCCGGCCC | TCGAGT | 81.4721884 | 4.01289051 |
| 49 | 49 | 45894175 | -1 | GCAAGGCCAGTGGCTCCGCCG | CTGGGT | 86.9498758 | 21.1982526 |
| 50 | 50 | 45894206 | -1 | GGTGTCCTCCTTCGGGCCATG | CGGGGT | 86.481861 | 27.1848396 |
| 51 | 51 | 45894230 | -1 | AGTCTTTGTGTCGTTGCGGGG | GTGGGT | 89.5581395 | 20.8768619 |
| 52 | 52 | 45894234 | -1 | TTGGAGTCTTTGTGTCGTTGC | GGGGGT | 92.6659815 | 1.87857423 |
| 53 | 53 | 45894254 | -1 | GCTTTCTCCACCTCCTGTAGT | TGGAGT | 74.7958898 | 15.3497161 |
| 54 | 54 | 45894575 | -1 | CTGCTGTTGGTGCCGGAGCTG | GTGGGT | 75.9773506 | 1.96971761 |
| 55 | 55 | 45894742 | -1 | GAGGGCGAGGGGCGGCGGCGC | AGGGGT | 47.5758005 | 0.0980997 |
| 56 | 56 | 45894874 | 1 | GCCTGGAAAGGGACCTGAGCA | AGGGAT | 60.1540743 | 7.22059518 |
| 57 | 57 | 45894890 | 1 | GAGCAAGGGATGCACGCACGC | GTGAGT | 91.3048847 | 22.0823232 |
| 58 | 58 | 45894916 | 1 | TGCGCGCGTGTGTGTGTGCTG | GAGGGT | 60.2887872 | 17.8078756 |
| 59 | 59 | 45894946 | -1 | CAGCCTCCACCTGGGGTCTGC | GCGAAT | 65.5772768 | 1.77179237 |
| 60 | 60 | 45894956 | -1 | CCTGCCGGCACAGCCTCCACC | TGGCGT | 63.1187292 | 12.7127654 |
| 61 | 61 | 45894967 | 1 | CCCAGGTGGAGGCTGTGCCGG | CAGGGT | 66.989345 | 8.75241839 |
| 62 | 62 | 45895026 | -1 | TTGCGGCAAAAGGCTGCAGTC | GAGAGT | 74.4118029 | 4.65326729 |
| 63 | 63 | 45896090 | 1 | TGTGTGTGTGTGTGTGTGTGG | AGGGGT | 17.6160322 | 21.170253 |
| 64 | 64 | $5895115 | 1 | GTCCGATAACGACCCCCGAAA | CCGAAT | 97.3700561 | 22.352532 |
| 65 | 65 | 45895117 | -1 | GCGGATTTCAGATTCGGTTTC | GGGGGT | 90.6758258 | 1.14961599 |
| 66 | 66 | 45895138 | -1 | TGGCGAACAGCGGCAGGGACA | GCGGAT | 65.9105326 | 3.87917232 |
| 67 | 67 | 45895170 | 1 | GCCATCAGCTCTAAGAAAGAC | GTGGAT | 70.4287264 | 23.4249124 |
| 68 | 68 | 45895175 | 1 | CAGCTCTAAGAAAGACGTGGA | TCGGGT | 83.2675292 | 6.55577012 |
| 69 | 69 | 45887397 | -1 | GGACAACCATTCTGAGGACAT | CAGAGT | 73.7901057 | 10.1765611 |
| 70 | 70 | 45887630 | -1 | ACTGCACTACAGCCTGGACAA | CAGAGT | 57.392231 | 32.1426061 |
| 71 | 71 | 45887827 | -1 | GCAGGTGGATCACCTGAAGTC | AGGAGT | 79.2541241 | 4.62656733 |
| 72 | 72 | 15988066 | -1 | AGCTGGGAAGGACATGTGGGA | CTGAAT | 58.8196471 | 46.0320087 |
| 73 | 73 | 45888194 | -1 | TCCTGGCCATGAAATGTAAAC | TAGGGT | 80.0779966 | 34.8808509 |
| 74 | 74 | 48888384 | -1 | AGGTCAGAGCCCTTGTTGGGA | AAGGAT | 73.1531089 | 2.27725659 |
| 75 | 75 | 48888807 | 1 | ATGGTGACAGGAAGAGCAAAG | CGGGGT | 48.0069423 | 66.5501815 |
| 76 | 76 | 45888828 | -1 | GACTACTGAACTTAGGTTCAA | CTGAAT | 88.546618 | 12.5103283 |
| 77 | 77 | 45889058 | -1 | ATGATAAGGTGAGTTTTAGAG | CTGGAT | 66.6749579 | 65.9824787 |
| 78 | 78 | 45889239 | -1 | ACTGCACTTCTACCTGGGCAA | CAGAGT | 66.0161798 | 52.8030303 |
| 79 | 79 | 45889443 | -1 | CACTTTGGGAGGCAGAGGTAG | GTGGAT | 50.3759205 | 26.4257077 |
| 80 | 80 | 45889659 | 1 | CCCAGTGTGGGGCCAACATGA | CTGGGT | 79.7054939 | 6.80723064 |
| 81 | 81 | 45889813 | 1 | GGAACAAGTCCTTCCCTATAG | GGGAAT | 86.8612417 | 24.8501093 |
| 82 | 82 | 48889978 | 1 | AGGGCTTTTATCATATTGCCA | TAGGGT | 81.8505267 | 57.2184178 |
| 83 | 83 | 45890204 | -1 | ATACTTTTTATGTGGGGGTGG | GGGGAT | 29.2430136 | 8.43231343 |
| 84 | 84 | 45890436 | -1 | GCTTGAGGCAGGGTCATCATT | TAGAGT | 81.4093508 | 21.7392466 |
| 85 | 85 | 45890861 | -1 | CAACTCTTCGAGCAGTCTTGG | GTGAGT | 85.2045243 | 32.6405628 |
| 86 | 86 | 45890768 | 1 | TCTAAGGTCATACAAGATGGC | TAGGAT | 87.0357152 | 29.7348071 |
| 87 | 87 | 45890974 | 1 | ACCTACATTGACTAAATTATC | TGGAAT | 77.4168613 | 6.69167391 |
| 88 | 88 | 45891168 | 1 | AAATGTGAAATTTGAATGTAG | ACGAGT | 53.0514588 | 14.4789416 |
| 89 | 89 | 45891388 | -1 | GTCCTGTATCCTGATTGATAC | AGGAAT | 86.586271 | 12.5791488 |
| 90 | 90 | 45891637 | 1 | TTTTTGTGATTTTAGTAGAGA | TGGGAT | 42.507869 | 2.50646081 |
| 91 | 91 | 48891855 | -1 | TTTACTCTTCCTTTCCGCCAC | CAGAAT | 89.9524073 | 44.2056074 |
| 92 | 92 | 45892082 | -1 | GGGACATTTCCAGTCTCTAGA | AGGAGT | 81.0469934 | 52.032287 |
| 93 | 93 | 45892213 | 1 | TTTGTAGGCAAAGGAAAACCT | CAGAAT | 59.9842427 | 53.8381039 |
| 94 | 94 | 45895406 | 1 | CTTTTACATATTTTTGAGCAA | GAGAGT | 50.1306495 | 68.8116789 |
| 95 | 95 | 45893605 | -1 | CTGTCTCAGCCTCCCAGCTAC | TGGGAT | 69.3861361 | 5.41526824 |
| 96 | 96 | 45892786 | 1 | CTTCTGAATACTGATCTAACT | AGGGGT | 75.9342922 | 5.49966728 |

Table 2 List of control sgRNA guide sequences.

TABLE 2

| SEQ ID No. | Control Guide | Sequence |
|---|---|---|
| 98 | 1 | ACGGAGGCTAAGCGTCGCAA |
| 99 | 2 | CGCTTCCGCGGCCCGTTCAA |
| 100 | 3 | GTAGGCGCGCCGCTCTCTAC |

### Example 2

(1) Experimental Methods

Cell Culture and Transfection

SK-N-AS (American Type Culture Collection) cells were seeded 24-72 hours prior to transfection in 12-well plates at a density of 75,000-200,000 cells per well and cultured in DMEM media supplemented with 10% FBS and 2 mM fresh L-glutamine, 1 mM sodium pyruvate and non-essential amino acids. Cells were transfected with 1000 ng sgRNA containing px601-CMV-dSaCas9-KRAB-P2A-Puro (modified from GenScript) plasmid using 3.0 μl of TransIT-VirusGEN (Mirus Bio), according to manufacturer's instructions. 24-36 hours following transfection, transfected cells were enriched by puromycin selection (1.5 μg/ml in DMEM). Cells were harvested at 72 h after transfection and lysed in RLT buffer (Qiagen) to extract total RNA using RNeasy kit (Qiagen).

Gene Expression Analysis

For Taqman analysis, max 1.5 μg of total RNA was used to generate cDNA using TaqMan High-Capacity RNA-to-cDNA Kit (Applied Biosystems) in 10 μl volume. The generated cDNA was diluted 10-fold and 3.33 μl was used per Taqman reaction. The Taqman primers and probes for the MAPT and HPRT gene were obtained from Applied Biosystems. Taqman reaction was run using Taqman gene expression master mix (ThermoFisher) in ThermoFisher QuantStudio 5 Real-Time PCR System and analyzed using QuantStudio 5 analysis software.

Taqman probe product IDs:

MAPT: Hs00902193_ml (FAM-MGB)

HPRT: Hs99999909_ml (VIC PL)

Taqman qPCR condition:

Step 1; 50° C. 2 min

Step 2; 95° C. 2 min

Step 3; 95° C. 1 sec

Step 4; 60° C. 20 sec

Repeat Steps 3 and 4; 45 times

Selection of sgRNA Sequence

The location of the guide RNA target sites relative to the MAPT gene is shown in FIG. 1. The selected guide RNA sequences (Table 3) were fused with the tracer RNA sequence to form single-molecule guide RNA (sgRNA) sequences, and were cloned into px601-CMV-dSaCas9-KRAB-P2A-Puro (modified from GenScript). The sgRNA expression is driven by the hU6 promoter, and the vector expresses the puromycin gene under a CMV/P2A promoter mechanism to facilitate tracking and selection of the sgRNA expressing cells. Three control sgRNA guides (Table 2) were selected from the Human CRISPR Knockout Pooled Library (Sanjana N. E. et al, Nature Methods, 11(8), p. 783, 2014).

(2) Results

Suppression of MAPT Gene Expression by the RNP

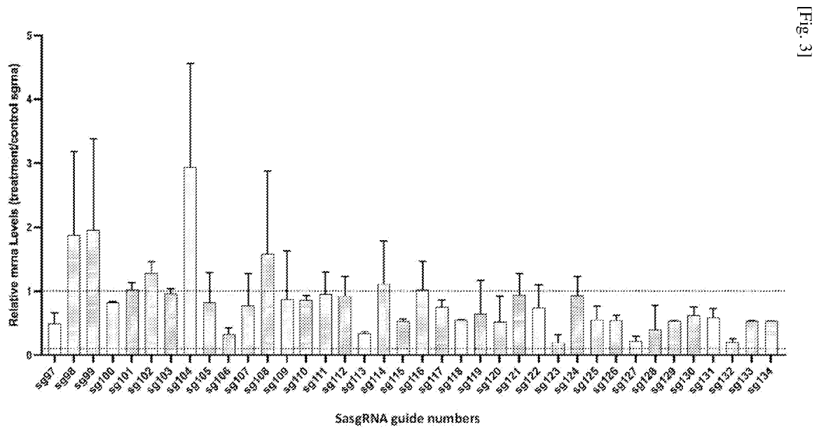

The suppression of MAPT transcript by the additional thirty-eight sgRNAs are shown (FIG. 3), where MAPT transcript expression was normalized to the mRNA levels of the HPRT gene. Detected MAPT expression levels after transfection with control sgRNA guides were set to 1.0. sgRNA #123, 127 and 132 showed close to 80% suppression whereas 113 and 106 showed 70% suppression. The experiments detailed were conducted at least three times, and the mean-fold suppression values and standard deviations are shown.

Table 3 List of sa sgRNA guides (with NNGRRT PAM sequence) within ‘UCSC Genome Browser on Human December 2013 (GRCh38/hg38) Assembly; chromosome 17: 45,887,381-45,962,898’.

| SEQ ID | Guide RNA | | | | | Specificity | Efficiency |
|---|---|---|---|---|---|---|---|
| NO. | (sa sgRNA) | Position | Strand | Sequence | PAM | Score | Score |
| 118 | 97 | 45895206 | -1 | AGGTGCAGGGAGGGGCGTGCA | GGGAGT | 59.309655 | 4.73320807 |
| 119 | 98 | 45895244 | -1 | CAGGGGCAGTGAAGGCCCTGT | CGGAAT | 69.6206557 | 32.383784 |
| 120 | 99 | 45895315 | -1 | CCCAGCCCCCAAATGTCCCCT | ACGGGT | 77.3756342 | 15.4724932 |
| 121 | 100 | 45895349 | 1 | GGAGAAATCGAGGAGATGGGG | AGGGGT | 48.0823599 | 19.4042152 |
| 122 | 101 | 45895438 | 1 | CGCCGTGCCTGAGAACAGTGC | GCGGAT | 80.2954811 | 20.1550618 |
| 123 | 102 | 45895454 | -1 | TGCCTTTGCGAGCGTGCACAG | TGGGAT | 87.1186343 | 41.3694288 |
| 124 | 103 | 45895467 | 1 | CACTGTGCACGCTCGCAAAGG | CAGGGT | 96.1500184 | 10.186814 |
| 125 | 104 | 46895510 | -1 | GCGAACGGGGACCAGCGGCCG | CCGAGT | 85.3329528 | 15.1055589 |
| 126 | 105 | 45895549 | 1 | CACGCACAGCCGCAGCCACGC | ACGGAT | 83.8131281 | 4.1878061 |
| 127 | 106 | 45895583 | 1 | GGGCTGCAGGTGCATCTCGGG | GCGGAT | 78.7301006 | 6.03387682 |
| 128 | 107 | 45895684 | 1 | GGCTCGCCCCTCACTGCGGCA | GTGGGT | 85.1877932 | 55.616967 |
| 129 | 108 | 45895706 | -1 | TCCTCCCCCTTCCTCGCCCAC | CAGGGT | 71.2852148 | 16.6218252 |
| 130 | 109 | 45895716 | 1 | CCCTGGTGGGCGAGGAAGGGG | GAGGAT | 41.7697132 | 2.35704489 |
| 131 | 110 | 45895751 | -1 | AAAAAAAGGGGGGCTGGGGGC | GGGAGT | 41.2972073 | 0.55408919 |
| 132 | 111 | 45895830 | 1 | TCTTTACGGTGCCATGCCAAA | CCGGGT | 88.5180082 | 54.2501452 |
| 133 | 112 | 45896030 | 1 | CTGGTTTCTGGCTTTTATTCT | GAGGGT | 45.8770507 | 0.89271586 |
| 134 | 113 | 45896037 | -1 | GGGAGGTTGACTGAACACCCT | CAGAAT | 84.4628587 | 37.0688894 |
| 135 | 114 | 45896107 | 1 | TCCTCATTTCCGAGCCCATTG | TTGGAT | 86.5360858 | 11.1535183 |
| 136 | 115 | 45896124 | | ATTGTTGGATCTCGAGGCTTG | CTGGGT | 90.2563231 | 4.75122805 |
| 137 | 116 | 45896139 | | GGCTTGCTGGGTTCGATGAAC | TCGAGT | 96.6052647 | 1.64593323 |
| 138 | 117 | 45896149 | | GCCGGGGGTCGGGGGGTTGAC | TCGAGT | 81.9105554 | 2.00201999 |
| 139 | 118 | 45896159 | | TTCCATGCGTGCCGGGGGTCG | GGGGGT | 91.9530435 | 1.44849023 |
| 140 | 119 | 45896167 | | CACGCCCGTTCCATGCGTGCC | GGGGGT | 96.1743145 | 0.31699411 |
| 141 | 120 | 45892794 | | TACTGATCTAACTAGGGGTTG | CAGGGT | 89.6918865 | 9.65934561 |
| 142 | 121 | 45892881 | | ACCAAGATAGAGGTCTTGAAC | TAGGAT | 86.6149258 | 2.22133977 |
| 143 | 122 | 45892928 | | CAACAAAAAGTCAATTCCAGG | CTGAGT | 59.6496529 | 60.3179983 |
| 144 | 123 | 45892932 | | CATGAGCCACTGCACTCAGCC | TGGAAT | 62.8908565 | 9.07396863 |
| 145 | 124 | 45892965 | | CACCTCAGCCTCCCAAAGCGT | TGGGAT | 71.7665018 | 21.2102322 |
| 146 | 125 | 45892979 | | AACGCTTTGGGAGGCTGAGGT | GGGAGT | 50.6652373 | 9.9538773 |
| 147 | 126 | 45893095 | | CAATCTTCCCACCTCAGCCTT | CTGAGT | 65.0078225 | 25.505003 |
| 148 | 127 | 45893120 | | TGGGAAGATTGCTTGAGCCCC | AGGAGT | 81.4308321 | 1.44369449 |
| 149 | 128 | 45893172 | | GAGACAAGGTCTTGCTCAGGC | TGGAGT | 42.5862188 | 20.7088644 |
| 150 | 129 | 45893212 | | TGTACACCTTAGAAAAGTCAG | TGGAAT | 74.3622004 | 57.8656558 |
| 151 | 130 | 45893283 | | TCTGCCCTAGCCTTCTGACTA | CAGAAT | 81.7937919 | 12.1321814 |
| 152 | 131 | 45893341 | | GTGGTTCTCACTCCTACTTCT | TTGGGT | 71.6156126 | 3.48872191 |
| 153 | 132 | 45893341 | | GGGGCATATACCCAAAGAAGT | AGGAGT | 76.8761349 | 39.4682588 |
| 154 | 133 | 45893425 | | AAGGTCAGTTCCAGAAACTGT | GTGAAT | 67.2726045 | 17.7360205 |
| 155 | 134 | 45893485 | | AACATATGTTGATTTTTTAAA | AAGAAT | 22.7028856 | 17.540826 |

### INDUSTRIAL APPLICABILITY

According to the present invention, the expression of MAPT gene in human cells can be suppressed. Thus, the present invention is expected to be extremely useful for the treatment and/or prevention of AD.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is, therefore, to be understood that, within the scope of the appended claims, the invention may be practiced otherwise than as specifically described herein.

All patents and other references mentioned above are incorporated in full herein by this reference, the same as if set forth at length.

This application is based on U.S. provisional patent application No. 63/049,736 (filing date: Jul. 9, 2020), and U.S. provisional patent application No. 63/212,429 (filing date: Jun. 18, 2021), both filed in US, the contents of which are incorporated in full herein.

1. A polynucleotide, comprising the following base sequences:
(a) a base sequence encoding a fusion protein of a nuclease-deficient CRISPR effector protein and a transcription repressor, and
(b) a base sequence encoding a guide RNA targeting a continuous region of 18 to 24 nucleotides in length in a region set forth in SEQ ID NO: 54, 55, 56, 57, 68, 153 or 97 in the expression regulatory region of human MAPT gene.

2. The polynucleotide according to claim 1, wherein the base sequence encoding the guide RNA comprises the base sequence set forth in SEQ ID NO: 54, 55, 56, 57, 68, 153 or 97, or the base sequence set forth in SEQ ID NO: 54, 55, 56, 57, 68, 153 or 97 in which 1 to 3 bases are deleted, substituted, inserted, and/or added.

3. The polynucleotide according to claim 1, comprising at least two different base sequences encoding the guide RNA.

4. The polynucleotide according to claim 1, wherein the transcriptional repressor is selected from the group consisting of KRAB, MeCP2, SIN3A, HDT1, MBD2B, NIPP1, and HP1A.

5. The polynucleotide according to claim 4, wherein the transcriptional repressor is KRAB.

6. The polynucleotide according to claim 1, wherein the nuclease-deficient CRISPR effector protein is dCas9.

7. The polynucleotide according to claim 6, wherein the dCas9 is derived from Staphylococcus aureus.

8. The polynucleotide according to claim 1, further comprising a promoter sequence for the base sequence encoding the guide RNA and/or a promoter sequence for the base sequence encoding the fusion protein of the nuclease-deficient CRISPR effector protein and the transcriptional repressor.

9. The polynucleotide according to claim 8, wherein the promoter sequence for the base sequence encoding the guide RNA is selected from the group consisting of U6 promoter, SNR6 promoter, SNR52 promoter, SCR1 promoter, RPR1 promoter, U3 promoter, and H1 promoter.

10. The polynucleotide according to claim 9, wherein the promoter sequence for the base sequence encoding the guide RNA is U6 promoter.

11. The polynucleotide according to claim 8, wherein the promoter sequence for the base sequence encoding the fusion protein of the nuclease-deficient CRISPR effector protein and the transcriptional repressor is a ubiquitous promoter or a neuron specific promoter.

12. The polynucleotide according to claim 11, wherein the ubiquitous promoter is selected from the group consisting of EFS promoter, CMV promoter and CAG promoter.

13. A vector comprising a polynucleotide according to claim 1.

14. The vector according to claim 13, wherein the vector is a plasmid vector or a viral vector.

15. The vector according to claim 14, wherein the viral vector is selected from the group consisting of adeno-associated virus (AAV) vector, adenovirus vector, and lentivirus vector.

16. The vector according to claim 15, wherein the AAV vector is selected from the group consisting of AAV1, AAV2, AAV6, AAV7, AAV8, AAV9, Anc80, AAV₅₈₇MTP, AAV₅₈₈MTP, AAV-B1, AAVM41, and AAVrh74.

17. The vector according to claim 16, wherein the AAV vector is AAV9.

18-19. (canceled)

20. A method for treating or preventing Alzheimer's disease, comprising administering the vector of claim 13, to a subject in need thereof.


SEQUENCE LISTING (155 sequences)

SEQ ID NO: 1 - DNA, 21 bp, Homo sapiens
actgcactccagcctgggtga

SEQ ID NO: 2 - DNA, 21 bp, Homo sapiens
gtttcaccctgtcacccaggc

SEQ ID NO: 3 - DNA, 21 bp, Homo sapiens
attacaccactgcactccagc

SEQ ID NO: 4 - DNA, 21 bp, Homo sapiens
ctactggggaggctgaggcag

SEQ ID NO: 5 - DNA, 21 bp, Homo sapiens
tgcctcagcctccccagtagc

SEQ ID NO: 6 - DNA, 21 bp, Homo sapiens
ttattgtatttttagtagaga

SEQ ID NO: 7 - DNA, 21 bp, Homo sapiens
tgggtggatcacctgaggtca

SEQ ID NO: 8 - DNA, 21 bp, Homo sapiens
cactttgggaggccgaggtgg

SEQ ID NO: 9 - DNA, 21 bp, Homo sapiens
tcagcactttgggaggccgag

SEQ ID NO: 10 - DNA, 21 bp, Homo sapiens
cctggccgtcacctggtggtg

SEQ ID NO: 11 - DNA, 21 bp, Homo sapiens
agatgtaaataacgcttgggc

SEQ ID NO: 12 - DNA, 21 bp, Homo sapiens
cttgggcaggaatatggagca

SEQ ID NO: 13 - DNA, 21 bp, Homo sapiens
caggaatatggagcacgggat

SEQ ID NO: 14 - DNA, 21 bp, Homo sapiens
gatgggcggccaactgttaga

SEQ ID NO: 15 - DNA, 21 bp, Homo sapiens
aggagtgttggggggcagagt

SEQ ID NO: 16 - DNA, 21 bp, Homo sapiens
gttctgaggagtgttgggggg

SEQ ID NO: 17 - DNA, 21 bp, Homo sapiens
aagaggagaggataagttctg

SEQ ID NO: 18 - DNA, 21 bp, Homo sapiens
tcacctggggaaagaagagga

SEQ ID NO: 19 - DNA, 21 bp, Homo sapiens
ttccccaggtgaactttgaac

SEQ ID NO: 20 - DNA, 21 bp, Homo sapiens
ccaggatggctgagccccgcc

SEQ ID NO: 21 - DNA, 21 bp, Homo sapiens
tggaagatcacgctgggacgt

SEQ ID NO: 22 - DNA, 21 bp, Homo sapiens
tacaccatgcaccaagaccaa

SEQ ID NO: 23 - DNA, 21 bp, Homo sapiens
ggcccagatcactgcaagcca

SEQ ID NO: 24 - DNA, 21 bp, Homo sapiens
gtggcgggaacagtttgcatc

SEQ ID NO: 25 - DNA, 21 bp, Homo sapiens
atttaaaatttctttgcaatt

SEQ ID NO: 26 - DNA, 21 bp, Homo sapiens
gtaaagtaaagcctcattaat

SEQ ID NO: 27 - DNA, 21 bp, Homo sapiens
gtctggccattatctcactgc

SEQ ID NO: 28 - DNA, 21 bp, Homo sapiens
tgcctgggccttccaaagtgc

SEQ ID NO: 29 - DNA, 21 bp, Homo sapiens
cactttggaaggcccaggcag

SEQ ID NO: 30 - DNA, 21 bp, Homo sapiens
ggtctcaaattcctggcctca

SEQ ID NO: 31 - DNA, 21 bp, Homo sapiens
gcaggaggatcccttgaggcc

SEQ ID NO: 32 - DNA, 21 bp, Homo sapiens
atttttaaattattttagaga

SEQ ID NO: 33 - DNA, 21 bp, Homo sapiens
atgtctatagtcctagctact

SEQ ID NO: 34 - DNA, 21 bp, Homo sapiens
tgatccttctgcctcagcatc

SEQ ID NO: 35 - DNA, 21 bp, Homo sapiens
ctactcaggatgctgaggcag

SEQ ID NO: 36 - DNA, 21 bp, Homo sapiens
gcagaaggatcacttgagccc

SEQ ID NO: 37 - DNA, 21 bp, Homo sapiens
agtgggatgatttctatgtag

SEQ ID NO: 38 - DNA, 21 bp, Homo sapiens
atcaagtttaagcccaagcag

SEQ ID NO: 39 - DNA, 21 bp, Homo sapiens
gccccaggcttcggccttagc

SEQ ID NO: 40 - DNA, 21 bp, Homo sapiens
gcctggggcctgggcgaccag

SEQ ID NO: 41 - DNA, 21 bp, Homo sapiens
gctgtaaatagagcttgaagt

SEQ ID NO: 42 - DNA, 21 bp, Homo sapiens
gaaaaaaaaatcttaaattag

SEQ ID NO: 43 - DNA, 21 bp, Homo sapiens
tgtacttaaaaaaaaagaaac

SEQ ID NO: 44 - DNA, 21 bp, Homo sapiens
tacagttctactgtattgtaa

SEQ ID NO: 45 - DNA, 21 bp, Homo sapiens
tttaagccgatttgttaagga

SEQ ID NO: 46 - DNA, 21 bp, Homo sapiens
ccttttttgttactgaccaag

SEQ ID NO: 47 - DNA, 21 bp, Homo sapiens
gggggcggtttcgggactacg

SEQ ID NO: 48 - DNA, 21 bp, Homo sapiens
ggccttccacgtggccggccc

SEQ ID NO: 49 - DNA, 21 bp, Homo sapiens
gcaaggccagtggctccgccg

SEQ ID NO: 50 - DNA, 21 bp, Homo sapiens
ggtgtcctccttcgggccatg

SEQ ID NO: 51 - DNA, 21 bp, Homo sapiens
agtctttgtgtcgttgcgggg

SEQ ID NO: 52 - DNA, 21 bp, Homo sapiens
ttggagtctttgtgtcgttgc

SEQ ID NO: 53 - DNA, 21 bp, Homo sapiens
gctttctccacctcctgtagt

SEQ ID NO: 54 - DNA, 21 bp, Homo sapiens
ctgctgttggtgccggagctg

SEQ ID NO: 55 - DNA, 21 bp, Homo sapiens
gagggcgaggggcggcggcgc

SEQ ID NO: 56 - DNA, 21 bp, Homo sapiens
gcctggaaagggacctgagca

SEQ ID NO: 57 - DNA, 21 bp, Homo sapiens
gagcaagggatgcacgcacgc

SEQ ID NO: 58 - DNA, 21 bp, Homo sapiens
tgcgcgcgtgtgtgtgtgctg

SEQ ID NO: 59 - DNA, 21 bp, Homo sapiens
cagcctccacctggggtctgc

SEQ ID NO: 60 - DNA, 21 bp, Homo sapiens
cctgccggcacagcctccacc

SEQ ID NO: 61 - DNA, 21 bp, Homo sapiens
cccaggtggaggctgtgccgg

SEQ ID NO: 62 - DNA, 21 bp, Homo sapiens
ttgcggcaaaaggctgcagtc

SEQ ID NO: 63 - DNA, 21 bp, Homo sapiens
tgtgtgtgtgtgtgtgtgtgg

SEQ ID NO: 64 - DNA, 21 bp, Homo sapiens
gtccgataacgacccccgaaa

SEQ ID NO: 65 - DNA, 21 bp, Homo sapiens
gcggatttcagattcggtttc

SEQ ID NO: 66 - DNA, 21 bp, Homo sapiens
tggcgaacagcggcagggaca

SEQ ID NO: 67 - DNA, 21 bp, Homo sapiens
gccatcagctctaagaaagac

SEQ ID NO: 68 - DNA, 21 bp, Homo sapiens
cagctctaagaaagacgtgga

SEQ ID NO: 69 - DNA, 21 bp, Homo sapiens
ggacaaccattctgaggacat

SEQ ID NO: 70 - DNA, 21 bp, Homo sapiens
actgcactacagcctggacaa

SEQ ID NO: 71 - DNA, 21 bp, Homo sapiens
gcaggtggatcacctgaagtc

SEQ ID NO: 72 - DNA, 21 bp, Homo sapiens
agctgggaaggacatgtggga

SEQ ID NO: 73 - DNA, 21 bp, Homo sapiens
tcctggccatgaaatgtaaac

SEQ ID NO: 74 - DNA, 21 bp, Homo sapiens
aggtcagagcccttgttggga

SEQ ID NO: 75 - DNA, 21 bp, Homo sapiens
atggtgacaggaagagcaaag

SEQ ID NO: 76 - DNA, 21 bp, Homo sapiens
gactactgaacttaggttcaa

SEQ ID NO: 77 - DNA, 21 bp, Homo sapiens
atgataaggtgagttttagag

SEQ ID NO: 78 - DNA, 21 bp, Homo sapiens
actgcacttctacctgggcaa

SEQ ID NO: 79 - DNA, 21 bp, Homo sapiens
cactttgggaggcagaggtag

SEQ ID NO: 80 - DNA, 21 bp, Homo sapiens
cccagtgtggggccaacatga

SEQ ID NO: 81 - DNA, 21 bp, Homo sapiens
ggaacaagtccttccctatag

SEQ ID NO: 82 - DNA, 21 bp, Homo sapiens
agggcttttatcatattgcca

SEQ ID NO: 83 - DNA, 21 bp, Homo sapiens
atactttttatgtgggggtgg

SEQ ID NO: 84 - DNA, 21 bp, Homo sapiens
gcttgaggcagggtcatcatt

SEQ ID NO: 85 - DNA, 21 bp, Homo sapiens
caactcttcgagcagtcttgg

SEQ ID NO: 86 - DNA, 21 bp, Homo sapiens
tctaaggtcatacaagatggc

SEQ ID NO: 87 - DNA, 21 bp, Homo sapiens
acctacattgactaaattatc

SEQ ID NO: 88 - DNA, 21 bp, Homo sapiens
aaatgtgaaatttgaatgtag

SEQ ID NO: 89 - DNA, 21 bp, Homo sapiens
gtcctgtatcctgattgatac

SEQ ID NO: 90 - DNA, 21 bp, Homo sapiens
tttttgtgattttagtagaga

SEQ ID NO: 91 - DNA, 21 bp, Homo sapiens
tttactcttcctttccgccac

SEQ ID NO: 92 - DNA, 21 bp, Homo sapiens
gggacatttccagtctctaga

SEQ ID NO: 93 - DNA, 21 bp, Homo sapiens
tttgtaggcaaaggaaaacct

SEQ ID NO: 94 - DNA, 21 bp, Homo sapiens
cttttacatatttttgagcaa

SEQ ID NO: 95 - DNA, 21 bp, Homo sapiens
ctgtctcagcctcccagctac

SEQ ID NO: 96 - DNA, 21 bp, Homo sapiens
cttctgaatactgatctaact

SEQ ID NO: 97 - DNA, 321 bp, Homo sapiens
cagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgc

SEQ ID NO: 98 - DNA, 20 bp, Artificial Sequence
  control non-targeting targeting sequence
acggaggctaagcgtcgcaa

SEQ ID NO: 99 - DNA, 20 bp, Artificial Sequence
  control non-targeting targeting sequence
cgcttccgcggcccgttcaa

SEQ ID NO: 100 - DNA, 20 bp, Artificial Sequence
  control non-targeting targeting sequence
gtaggcgcgccgctctctac

SEQ ID NO: 101 - RNA, 19 nt, Francisella novicid
  misc_feature (1)..(19): 5′-handle of crRNA
aauuucuacuguuguagau

SEQ ID NO: 102 - protein, 1053 aa, Staphylococcus aureus
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 103 - protein, 1053 aa, Staphylococcus aureus
  VARIANT (10)..(10): conversion of Asp residue into Ala residue
  VARIANT (557)..(557): conversion of His residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 104 - protein, 1028 aa, Artificial Sequence
  amino acid residues  (721st to 745th amino acid residues of dSaCas9) deletion mutant
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 105 - protein, 6 aa, Artificial Sequence
  GGSGGS linker
GGSGGS

SEQ ID NO: 106 - protein, 1034 aa, Artificial Sequence
  amino acid residues  (721st to 745th amino acid residues of dSaCas9) deletion mutant with GGSGGS linker
  VARIANT (10)..(10): conversion of Asp residue into Ala residue
  VARIANT (580)..(580): conversion of Asn residue into Ala residue
  MISC_FEATURE (721)..(726): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKGGSGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 107 - protein, 5 aa, Artificial Sequence
  SGGGS linker
SGGGS

SEQ ID NO: 108 - protein, 1033 aa, Artificial Sequence
  amino acid residues  (721st to 745th amino acid residues of dSaCas9) deletion mutant with SGGGS linker
  VARIANT (10)..(10): conversion of Asp residue into Ala residue
  VARIANT (580)..(580): conversion of Asn residue into Ala residue
  MISC_FEATURE (721)..(725): SGGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKSGGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 109 - protein, 886 aa, Artificial Sequence
  amino acid residues  (482nd to 648th amino acid residues of dSaCas9) deletion mutant
  VARIANT (10)..(10): conversion of Asp residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELARETRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 110 - protein, 892 aa, Artificial Sequence
  amino acid residues  (482nd to 648th amino acid residues of dSaCas9) deletion mutant with GGSGGS linker
  VARIANT (10)..(10): conversion of Asp residue into Ala residue
  MISC_FEATURE (482)..(487): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREGGSGGSTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 111 - DNA, 48 bp, Artificial Sequence
  DNA sequence encoding NLS
gccccaaagaagaagcggaaggtcggtatccacggagtcccagcagcc

SEQ ID NO: 112 - DNA, 48 bp, Artificial Sequence
  DNA sequence encoding NLS
aaaaggccggcggccacgaaaaaggccggccaggcaaaaaagaaaaag

SEQ ID NO: 113 - DNA, 3477 bp, Artificial Sequence
  dSaCas9 fused to KRAB (DNA)
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 114 - RNA, 21 nt, Homo sapiens
gagcaagggaugcacgcacgc

SEQ ID NO: 115 - DNA, 21 bp, Homo sapiens
gcgtgcgtgcatcccttgctc

SEQ ID NO: 116 - DNA, 19 bp, Francisella novicid
  misc_feature: 5′-handle of crRNA
aatttctactgttgtagat

SEQ ID NO: 117 - DNA, 60 bp, Staphylococcus aureus
  misc_feature: sequence encoding tracrRNA
gttttagtactctggaaacagaatctactaaaacaaggcaaaatgccgtgtttatctcgt

SEQ ID NO: 118 - DNA, 21 bp, Homo sapiens
aggtgcagggaggggcgtgca

SEQ ID NO: 119 - DNA, 21 bp, Homo sapiens
caggggcagtgaaggccctgt

SEQ ID NO: 120 - DNA, 21 bp, Homo sapiens
cccagcccccaaatgtcccct

SEQ ID NO: 121 - DNA, 21 bp, Homo sapiens
ggagaaatcgaggagatgggg

SEQ ID NO: 122 - DNA, 21 bp, Homo sapiens
cgccgtgcctgagaacagtgc

SEQ ID NO: 123 - DNA, 21 bp, Homo sapiens
tgcctttgcgagcgtgcacag

SEQ ID NO: 124 - DNA, 21 bp, Homo sapiens
cactgtgcacgctcgcaaagg

SEQ ID NO: 125 - DNA, 21 bp, Homo sapiens
gcgaacggggaccagcggccg

SEQ ID NO: 126 - DNA, 21 bp, Homo sapiens
cacgcacagccgcagccacgc

SEQ ID NO: 127 - DNA, 21 bp, Homo sapiens
gggctgcaggtgcatctcggg

SEQ ID NO: 128 - DNA, 21 bp, Homo sapiens
ggctcgcccctcactgcggca

SEQ ID NO: 129 - DNA, 21 bp, Homo sapiens
tcctcccccttcctcgcccac

SEQ ID NO: 130 - DNA, 21 bp, Homo sapiens
ccctggtgggcgaggaagggg

SEQ ID NO: 131 - DNA, 21 bp, Homo sapiens
aaaaaaaggggggctgggggc

SEQ ID NO: 132 - DNA, 21 bp, Homo sapiens
tctttacggtgccatgccaaa

SEQ ID NO: 133 - DNA, 21 bp, Homo sapiens
ctggtttctggcttttattct

SEQ ID NO: 134 - DNA, 21 bp, Homo sapiens
gggaggttgactgaacaccct

SEQ ID NO: 135 - DNA, 21 bp, Homo sapiens
tcctcatttccgagcccattg

SEQ ID NO: 136 - DNA, 21 bp, Homo sapiens
attgttggatctcgaggcttg

SEQ ID NO: 137 - DNA, 21 bp, Homo sapiens
ggcttgctgggttcgatgaac

SEQ ID NO: 138 - DNA, 21 bp, Homo sapiens
gccgggggtcggggggttgac

SEQ ID NO: 139 - DNA, 21 bp, Homo sapiens
ttccatgcgtgccgggggtcg

SEQ ID NO: 140 - DNA, 21 bp, Homo sapiens
cacgcccgttccatgcgtgcc

SEQ ID NO: 141 - DNA, 21 bp, Homo sapiens
tactgatctaactaggggttg

SEQ ID NO: 142 - DNA, 21 bp, Homo sapiens
accaagatagaggtcttgaac

SEQ ID NO: 143 - DNA, 21 bp, Homo sapiens
caacaaaaagtcaattccagg

SEQ ID NO: 144 - DNA, 21 bp, Homo sapiens
catgagccactgcactcagcc

SEQ ID NO: 145 - DNA, 21 bp, Homo sapiens
cacctcagcctcccaaagcgt

SEQ ID NO: 146 - DNA, 21 bp, Homo sapiens
aacgctttgggaggctgaggt

SEQ ID NO: 147 - DNA, 21 bp, Homo sapiens
caatcttcccacctcagcctt

SEQ ID NO: 148 - DNA, 21 bp, Homo sapiens
tgggaagattgcttgagcccc

SEQ ID NO: 149 - DNA, 21 bp, Homo sapiens
gagacaaggtcttgctcaggc

SEQ ID NO: 150 - DNA, 21 bp, Homo sapiens
tgtacaccttagaaaagtcag

SEQ ID NO: 151 - DNA, 21 bp, Homo sapiens
tctgccctagccttctgacta

SEQ ID NO: 152 - DNA, 21 bp, Homo sapiens
gtggttctcactcctacttct

SEQ ID NO: 153 - DNA, 21 bp, Homo sapiens
ggggcatatacccaaagaagt

SEQ ID NO: 154 - DNA, 21 bp, Homo sapiens
aaggtcagttccagaaactgt

SEQ ID NO: 155 - DNA, 21 bp, Homo sapiens
aacatatgttgattttttaaa